(19) United States
(12) Patent Application Publication
(10) Pub. No.: US 2025/0276957 A1
(43) Pub. Date: Sep. 4, 2025

(54) CRYSTALLINE FORMS OF AN IL-17 INHIBITOR

(71) Applicant: Novartis AG, Basel (CH)

(72) Inventors: Bo LIU, Shanghai (CN); Jiaqi SONG, Shanghai (CN); Yingcong ZHOU, Shanghai (CN)

(21) Appl. No.: 18/858,872

(22) PCT Filed: Apr. 21, 2023
(86) PCT No.: PCT/IB2023/054117

§ 371 Date: Oct. 22, 2024

(30) Foreign Application Priority Data
Apr. 25, 2022 (WO) PCT/CN2022/088928

(51) Int. Cl. C07D 401/12

(52) U.S. Cl. CPC C07D 401/12

(57) ABSTRACT

This application relates to crystalline forms of a small molecule IL-17 inhibitor.

### FIELD OF INVENTION

The present disclosure relates to crystalline forms of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide. The present disclosure also relates to a the use of the crystalline forms for the manufacture of a medicament the treatment of diseases and disorders which are typically ameliorated by the inhibition of IL-17. Such diseases and disorders may include autoimmune diseases and inflammatory conditions, in particular psoriasis.

### BACKGROUND

Polymorphism denotes the existence of more than one crystalline form of a substance.

Solid state form of the active pharmaceutical ingredient (API) of a particular drug is often an important determinant of the drug's ease of preparation, hygroscopicity, stability, solubility, storage stability, ease of formulation, rate of dissolution in gastrointestinal fluids and in vivo bioavailability. Crystalline forms occur where the same composition of matter crystallizes in a different lattice arrangement resulting in different thermodynamic properties and stabilities specific to the particular crystalline form. Crystalline forms may also include different hydrates or solvates of the same compound. In deciding which form is preferable, the numerous properties of the forms are compared and the preferred form chosen based on the many physical property variables. It is entirely possible that one form can be preferable in some circumstances where certain aspects such as ease of preparation, stability, etc. are deemed to be critical. In other situations, a different form may be preferred for greater dissolution rate and/or superior bioavailability.

Therefore, this ability of a chemical substance to crystallize in more than one crystalline form can have a profound effect on the shelf life, solubility, formulation properties, and processing properties of a drug. In addition, the action of a drug can be affected by the polymorphism of the drug molecule. Different polymorphs can have different rates of uptake in the body, leading to lower or higher biological activity than desired. In extreme cases, an undesired polymorph can even show toxicity. The occurrence of an unknown crystalline form during manufacture can have a significant impact.

It is not yet possible to predict whether a particular compound or salt of a compound will form polymorphs, whether any such polymorphs will be suitable for commercial use in a therapeutic composition, or which polymorphs will display such desirable properties. However, understanding which crystalline forms of a drug are possible in certain cases allows researchers to maximize the desired properties of a compound, such as solubility, formulation properties, processing properties, and shelf life. Understanding these factors early in the development of a new drug may mean a more active, more stable, or more cheaply manufactured drug.

### SUMMARY

The compound 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide is an inhibitor of Interleukin-17 (IL-17). The IL-17 family cytokines are important regulators of inflammatory responses and participate in immune responses to infections. The family consists of six members, IL-17A to IL-17F. IL-17A (frequently cited as IL-17), the prototypic member of the family, was originally identified as a cytokine produced by the subset of CD4+ T-cells that is now termed Th17 (Matsuzaki et al Microbiol. Immunol. 2018, 62, 1-13).

IL-17, a T-cell derived cytokine present e.g. in rheumatoid arthritis (RA), acts as a pro-inflammatory cytokine, particularly in conjunction with IL-1 and TNF-α (Chabaud et al Arthritis Rheum. 1999, 42, 963-970; Awane M et al J. Immunol. 1999, 162, 5337-5344). IL-17 induces MMP production and downregulates TIMP (Jovanovic et al J. Rheumatol. 2001, 28, 712-718), and blockage of IL-1 and IL-17 has a synergistic effect on inflammation and bone destruction in vivo (Chabaud et al Arthritis Rheum 2001, 44, 1293-1303). Inappropriate or excessive production of IL-17 is associated with the pathology of various diseases and disorders, such as rheumatoid arthritis (Witowski et al Cell. Mol. Life Sci. 2004, 61, 567-579), osteoarthritis, loosening of bone implants, acute transplant rejection (Antonysamy et al J. Immunol. 1999, 162, 577-584; Van Kooten et al J. Am. Soc. Nephrol. 1998, 9, 1526-1534), septicemia, septic or endotoxic shock, allergies, asthma (Molet et al J. Allergy Clin. Immunol. 2001, 108, 430-438), bone loss, psoriasis (Teunissen et al J. Invest. Dermatol. 1998, 111, 645-649), ischemia, systemic sclerosis (Kurasawa et al Arthritis Rheum. 2000, 43, 2455-2463), stroke, and other inflammatory disorders. Antibodies to IL-17 have been proposed for use in the treatment of IL-17 mediated diseases and disorders; see for instance, WO 95/18826 or WO 2006/013107. One of the major problems associated with such antibody-based treatments is that they require repeated subcutaneous injections. Such injections may be associated with pain, bruising of the injected area, skin irritation, or skin infection. Small molecule IL-17 modulators are described in WO2020/127685, Liu et al Sci. Rep. 2016, 6, 30859, WO 2013/116682 and WO2014/066726.

There is a need to provide a solid state form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide, which possesses physicochemical properties allowing for reliable production, storage of the bulk product, and/or reliable production of a safe and efficacious drug product comprising 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide.

2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide is described in Example 1 of presently unpublished patent application PCT/IB2021/060092, filed Nov. 1, 2021, which claims priority to unpublished European Patent application, number 20205121.5, filed Nov. 2, 2020, both of which are incorporated by reference in their entirety. As described in PCT/IB2021/060092, 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide is an IL-17 inhibitor having the structure of Formula (I):

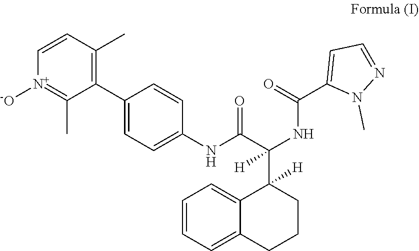

The activity of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide is described in PCT/IB2021/060092 as assessed by in vitro methods, such that said compound exhibits valuable pharmacological properties, and is therefore indicated for therapy related to IL-17. The cellular and TR-FRET assays described in PCT/IB2021/060092 and the data obtained in respect of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide are provided as follows:

### Human IL-17A x IL-17RA TR-FRET Assay

Dilutions of test compounds in DMSO are plated onto white low-volume 384-well plates (Greiner, #784075). Specifically, solutions of graded concentrations (5 mM to 150 nM; sixteen 2-fold dilution steps) are dispensed at 0.2 μL/well to yield final assay concentrations in the range of 100 μM to 3 nM. Each compound concentration is tested in duplicate.

All components of the assay are diluted in PBS containing 0.05% Tween20 (Sigma, #P1379, 5% stock in water), 1 mM EDTA (Invitrogen, #15575-038; 0.5M stock, pH8) and 0.05% bovine serum albumin (Roche; 5% stock in water). The final total assay volume is 10 μL. Reagents are added using a Multidrop Combi device (ThermoFisher, #5840320); between reagents, the instrument is rinsed with buffer:
(i) Biotinylated human IL-17A (PN106276; stock solution: 55.87 μM) is added at 5 μL/well from a 10 nM intermediate dilution to yield a final assay concentration of 5 nM. Each assay plate contains “low controls” where buffer is added in place of IL-17A.
(ii) After 30 minutes of incubation at RT, 2.5 μL/well of AF647-labeled human IL-17RA (PN110235; stock: 2.91 M) is added from a 120 nM intermediate solution, to yield a final assay concentration of 30 nM.
(iii) Following 30 minutes of incubation at RT, 2.5 μL/well of Eu-Streptavidin (PerkinElmer, #AD0062; stock: 500 μg/mL=8.3 μM) is added from an intermediate dilution of 4 nM to yield a final concentration of 1 nM.

After 30 to 60 minutes of incubation, FRET is measured in an EnVision 2101 Multilabel-Reader (Perkin Elmer) with excitation at 320 nm, and emission at 615 and 665 nm (program “TR-FRET Screening”, instrument: NIBR-00013163).

Each plate contains test compounds in columns 1-22 and controls in columns 23 and 24. The neutral control (NC) contains all the assay components, but DMSO instead of test compound. The active control (AC) mimics complete inhibition of the IL-17 ligand interaction, which is achieved by replacing the biotinylated cytokine with buffer. The fluorescence values for emission at 615 nm and 665 nm are loaded to the evaluation software Helios. In a normalization step, reader values are converted to % activity using the formula:

$$\% activity = - {100}^{*} ( x - NC ) / ( AC - NC )$$

where NC and AC are the median values of the neutral and active control wells on the same plate.

From the activity data at each compound concentration, the IC50 values are obtained using the 4-parameter logistic function (“Hill-slope model”) of Helios.

### IL-6 Release From NHDF Cells (Normal Human Dermal Fibroblasts)

NHDF cells isolated from normal adult mesoderm, connective tissues were cultured in fibroblast growth medium containing 2% supplement. The cells were detached from plastic using an accutase solution, washed and were seeded in 96-well plates at a density of 5×103/well in fibroblast growth medium lacking the supplements. The cells were allowed to adhere overnight before they were incubated with the compound/cytokine cocktail.

Compound stock solutions (10 mM in DMSO) were serially diluted in 96-well plates in fibroblast growth medium containing 0.2% supplement to give a maximal assay concentration of 10 μM. Diluted compounds were pre-incubated with recombinant human IL-17AA or IL-17AF plus human TNF-α for 30 minutes at room temperature on a shaker. NHDF cells were re-suspended in fibroblast growth medium/0.2% supplement (100 μl/well) followed by addition of the cytokine/compound mix to the cells (100 μl/well) to give a final compound starting concentration of 10 μM. The cells were incubated with the compound/cytokines cocktail for 24 hours at 37° C. (5% CO2). During the culture period, the final concentrations of IL-17AA were 30 pM, IL-17AF were 250 pM, and TNF-α were 6 pM. The final DMSO or plasma concentrations were 0.1% or 20%, respectively. All compound incubations were done in duplicate. The supernatants were collected to quantify IL-6 by ELISA according to the manufacturer's instructions. The optical density was measured at 450 nm by an EnVision Multilabel Plate Reader (Perkin Elmer). The IL-6 levels produced after TNF-α stimulation were subtracted from the IL-6 produced after IL-17/TNF-α co-stimulation. These IL-17-specific IL-6 concentrations were used for IC50 value calculation. IC50 values for inhibitors were then determined using sigmoidal dose-response curve fitting model (Excel Xlfit, model 205).

The potency of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide in the cell and TR-FRET assays is presented in the following table:

| | IL17AA + TNF | IL17AA FRET/IL17Rawt |
|---|---|---|
| Compound Name | IC50 IL-6 [umol I-1] | IC50 [umol I-1] |
| 2,4-dimethyl-3-(4-((S)-2-(1- | 0.004 | 0.008 |
| methyl-1H-pyrazole-5- | | |
| carboxamido)-2-((S)-1,2,3,4- | | |
| tetrahydronaphthalen-1- | | |
| yl)acetamido)phenyl)pyridine | | |
| 1-oxide | | |

PCT/IB2021/060092 provides no information about crystalline forms of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide. The preparation method of Example 1 described in PCT/IB2021/060092 provides an amorphous form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide, as demonstrated by the XRPD spectrum shown in FIG. 1.

In one aspect, the present invention provides a crystalline form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide in a free form (i.e. a non-salt form). In a particular embodiment, the crystalline form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide in a free form is anhydrous. In an embodiment, the crystalline form includes the form designated herein as Form A.

In another aspect, the present invention provides two forms of a crystalline form of the free form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide which are hydrate forms. In an embodiment, the two hydrate forms include the forms designated herein as H_{A} and H_{B}.

The embodiments designated herein as Form A, H_{A} and H_{B} are names used herein to identify a specific form, e.g. “Form A” or “Form H_{A}” or “Form H_{B}”, and should not be considered limiting with respect to any other substance possessing similar or identical physical and chemical characteristics, but rather it should be understood that these designations are mere identifiers that should be interpreted according to the characterization information also presented herein.

Preferably, crystalline Form A is substantially pure. More preferably, Form A is substantially phase pure.

In one aspect, the present invention also provides the use of crystalline Form A, H_{A} or H_{B} of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide for the preparation of a medicament. The medicament is for the treatment of disorders in a subject in need thereof, wherein the disorders are mediated by IL-17 or ameliorated by the inhibition of IL-17, in particular IL-17A and/or IL-17F. In particular, the mentioned use is of crystalline Form A. The disease mediated by IL-17 or ameliorated by the inhibition of IL-17, in particular IL-17A and/or IL-17F, is in particular, psoriasis. The medicament is, in particular, formulated for topical application. Accordingly, crystalline forms of the compound of Formula (I) as described herein are useful in the preparation of a topical medicament. The topical medicament is in particular useful for the treatment of psoriasis.

### BRIEF DESCRIPTION OF THE DRAWINGS

FIG. 1 provides an XRPD spectrum for amorphous form of compound of Formula (I), showing degrees 2θ (2-theta) on the X-axis and relative intensity on the Y-axis.

FIG. 2 provides an XRPD spectrum for an anhydrous crystalline form of compound of Formula (I), designated herein as Form A, showing degrees 2θ (2-theta) on the X-axis and relative intensity on the Y-axis.

FIG. 3 provides an DSC for an anhydrous crystalline form of the compound of Formula (I), designated herein as Form A.

FIG. 4 provides an TGA for an anhydrous crystalline form of the compound of Formula (I), designated herein as Form A.

FIG. 5 provides an XRPD spectrum for a hydrate crystalline form of compound of Formula (I), designated herein as Form H_{A}, showing degrees 2θ (2-theta) on the X-axis and relative intensity on the Y-axis.

FIGS. 6A and 6B provide DSC thermograms for a hydrate crystalline form of the compound of Formula (I), designated herein as Form H_{A}.

FIGS. 7A and 7B provides TGA graphs for a hydrate crystalline form of the compound of Formula (I), designated herein as Form H_{A}.

FIG. 8 provides an XRPD spectrum for a hydrate crystalline form of compound of Formula (I), designated herein as Form H_{B}, showing degrees 2θ (2-theta) on the X-axis and relative intensity on the Y-axis.

FIG. 9 provides an DSC for a hydrate crystalline form of the compound of Formula (I), designated herein as Form H_{B}.

FIG. 10 provides an TGA for a hydrate crystalline form of the compound of Formula (I), designated herein as Form H_{B}.

More detailed listings of the XRPD peaks for each of forms A, H_{A} and H_{B} are set forth in Tables 1, 2 and 3, respectively below, in which the % relative intensity (I/I₀×100) is also provided. It should be understood that in the X-ray powder diffraction spectra or pattern that there is inherent variability in the values measured in degrees 2θ (° 2θ) as a result of, for example, instrumental variation (including differences between instruments). As such, it should be understood that there is a variability of up to ±0.2° 2θ in XRPD peak measurements and yet such peak values would still be considered to be representative of a particular solid state form of the crystalline materials described herein. It should also be understood that other measured values from XRPD experiments and DSC/TGA experiments, such as relative intensity and water content, can vary as a result of, for example, sample preparation and/or storage and/or environmental conditions, and yet the measured values will still be considered to be representative of a particular solid state form of the crystalline materials described herein.

### DETAILED DESCRIPTION OF THE DISCLOSURE

### Definition

As used herein, the terms “about” and “substantially” indicate with respect to features such as endotherms, endothermic peak, exotherms, baseline shifts, temperature, etc., that their values can vary. With reference to X-ray diffraction peak positions, “about” or “substantially” means that typical peak position and intensity variability are taken into account. For example, one skilled in the art will appreciate that the peak positions (2θ) will show some inter-apparatus variability, typically as much as 0.2°. Occasionally, the variability could be higher than 0.2° depending on apparatus calibration differences. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. For DSC, variation in the temperatures observed will depend upon the rate of temperature change as well as sample preparation technique and the particular instrument employed. Thus, the endotherm/melting point values reported herein relating to DSC/TGA thermograms can vary ±5° C. (and still be considered to be characteristic of the particular crystalline form described herein). When used in the context of other features, such as, for example, percent by weight (% by weight), reaction temperatures, the term “about” indicates a variance of ±5%.

The terms “crystalline form(s)” or “crystalline modification(s)” or “polymorphic form(s)” or “polymorph(s)” will be used interchangeably herein. As used herein “polymorph” refers to crystalline forms having the same chemical composition but different spatial arrangements of the molecules, atoms, and/or ions forming the crystal. Each polymorph differs with respect to thermodynamic stability, physical parameters, x-ray structure and methods of preparation.

As used herein “amorphous” refers to a solid form of a molecule, atom, and/or ions that is not crystalline. An amorphous solid does not display a definitive X-ray diffraction pattern.

As used herein, “substantially pure,” when used in reference to a form, means a compound having a purity greater than 90 weight %, including greater than 90, 91, 92, 93, 94, 95, 96, 97, 98, and 99 weight %, and also including equal to about 100 weight % of Compound of Formula (I), based on the weight of the compound. The remaining material comprises other form(s) of the compound, and/or reaction impurities and/or processing impurities arising from its preparation. For example, a crystalline form of Compound of Formula (I) may be deemed substantially pure in that it has a purity greater than 90 weight %, as measured by means that are at this time known and generally accepted in the art, where the remaining less than 10 weight % of material comprises other form(s) of Compound of Formula (I) and/or reaction impurities and/or processing impurities.

As used herein, “substantially phase pure,” when used in reference to any crystalline form of the compound of Formula (I), means a compound having a phase purity of greater than about 90% by weight, including greater than about 90, 91, 92, 93, 94, 95, 96, 97, 98, and about 99% by weight, and also including equal to about 100% by weight of the compound of Formula (I), based on the weight of the compound on an anhydrous basis. The term “phase pure” or “phase purity” herein refers to phase homogeneity with respect to a particular solid state form of the compound of Formula (I) and does not necessarily imply a high degree of chemical purity absent an express statement to that effect. Phase purity may be determined according to methods known in the art, for example, using XRPD to do quantitative phase analysis using one or more approaches known in the art, for example, via an external standard method, direct comparisons of line (peak) characteristics which are attributed to different phases in a particular spectra, or via an internal standard method. However XRPD quantification of phase purity can be complicated by the presence of amorphous material. Accordingly, other methods that may be useful for determining phase purity include, for example, solid state NMR spectroscopy, Raman and/or infrared spectroscopy. One of skilled in the art would readily understand these methods and how to employ these additional (or alternative) methods for determining phase purity.

As used herein, “substantially chemically pure” when used in reference to any crystalline form of the compound of Formula (I), means a compound having a chemical purity greater than about 90% by weight, including greater than about 90, 91, 92, 93, 94, 95, 96, 97, 98, and about 99% by weight, and also including equal to about 100% by weight of the compound of Formula (I), based on the weight of the compound on an anhydrous basis. The remaining material generally comprises other compounds, such as for example, other stereoisomers of the compound of Formula (I), reaction impurities, starting materials, reagents, side products, solvents and/or other processing impurities arising from the preparation and/or isolation and/or purification of the particular crystalline form. For example, a crystalline form of the compound of Formula (I) may be deemed to be substantially chemically pure if it has been determined to have a chemical purity of greater than about 90% by weight, as measured by standard and generally accepted methods known in the art, where the remaining less than about 10% by weight constitutes other materials such as other stereoisomers of the compound of Formula (I), reaction impurities, starting materials, reagents, side products, solvents and/or processing impurities. Chemical purity may be determined according to methods known in the art, for example, high performance liquid chromatography (HPLC), LC-MS (liquid chromatography-mass spectrometry), nuclear magnetic resonance (NMR) spectroscopy, or infrared spectroscopy. One of skill in the art would readily understand these methods and how to employ these additional (or alternative) methods for determining chemical purity.

As used herein, the term “seed” can be used as a noun to describe one or more crystals of a crystalline compound of Formula (I). The term “seed” can also be used as a verb to describe the act of introducing said one or more crystals of a crystalline compound of Formula (I) into an environment (including, but not limited to e.g., a solution, a mixture, a suspension, or a dispersion) thereby resulting in the formation of more crystals or the growth of the introduced crystals of the crystalline compound of Formula (I).

As used herein, the term “subject” refers to an animal. Preferably, the animal is a mammal. A subject refers to for example, primates (e.g. humans), cows, sheep, goats, horses, dogs, cats, rabbits, rats, mice, fish, birds and the like. In a preferred embodiment, the subject is a human.

As used herein, a subject is “in need of” or “in need thereof” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both. In one embodiment, “treat” or “treating” refers to delaying the progression of the disease or disorder.

As used herein, the term “prevent”, “preventing” or “prevention” of any disease or disorder refers to the prophylactic treatment of the disease or disorder; or delaying the onset of the disease or disorder.

The term “comprising” encompasses “including” as well as “consisting”; e.g., a composition comprising X may consist exclusively of X or may include additional, e.g. X and Y.

### Crystalline Forms

The present disclosure relates to a crystalline form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide (the compound of Formula (I)), which is described and characterized herein.

In one embodiment, the present disclosure provides an anhydrous crystalline form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide.

In one embodiment, the present disclosure provides a crystalline form of 2,4-dimethyl-3-(4-((S)-2-(1-methyl-1H-pyrazole-5-carboxamido)-2-((S)-1,2,3,4-tetrahydronaphthalen- 1-yl)acetamido)phenyl)pyridine 1-oxide (Form A) having an X-ray powder diffraction (XRPD) pattern comprising a representative peak, in terms of 2θ, at 18.8±0.2° 2θ measured at a temperature of about 23° C. In another embodiment, the XRPD pattern further comprises one or more additional representative peaks chosen from 4.9±0.2° 2θ and 12.0±0.2° 2θ. In one aspect of the previous embodiment, the XRPD pattern further comprises one or more additional representative peaks chosen from 11.5±0.2° 2θ and 25.9±0.2° 2θ. In one aspect of the previous embodiment, the XRPD pattern for the crystalline Form A of the compound of Formula (I) may further comprise one, two, three, or four representative peaks selected from 15.5±0.2° 2θ, 19.5±0.2° 2θ, 20.5±0.2° 2θ, 22.1±0.2° 2θ and 23.4±0.2° 2θ. The XRPD pattern for the crystalline Form A of the compound of Formula (I) may comprise one or more representative peaks selected from 4.9±0.2° 2θ, 11.5±0.2° 2θ, 12.0±0.2° 2θ, 15.5±0.2° 2θ, 18.8±0.2° 2θ, 19.5±0.2° 2θ, 20.5±0.2° 2θ, 22.1±0.2° 2θ, 23.4±0.2° 2θ and 25.9±0.2° 2θ, measured at a temperature of about 23° C. The XRPD pattern for the crystalline Form A may comprise one or more (e.g. two, three, four, five or six) representative peaks selected from the peaks disclosed in Table 1 and measured at a temperature of about 23° C.

In another aspect of the above embodiment, the crystalline Form A of compound of Formula (I) is characterized by a x-ray powder diffraction pattern comprising three or more 2θ values selected from the group consisting of 4.9±0.2° 2θ, 11.5±0.2° 2θ, 12.0±0.2° 2θ, 15.5±0.2° 2θ, 18.8±0.2° 2θ, 19.5±0.2° 2θ, 20.5±0.2° 2θ, 22.1±0.2° 2θ, 23.4±0.2° 2θ and 25.9±0.2° 2θ, measured at a temperature of about 23° C.

In another aspect of the above embodiment, the crystalline Form A of compound of Formula (I) is characterized by a x-ray powder diffraction pattern comprising four or more 2θ values selected from the group consisting of 4.9±0.2° 2θ, 11.5±0.2° 2θ, 12.0±0.2° 2θ, 15.5±0.2° 2θ, 18.8±0.2° 2θ, 19.5±0.2° 2θ, 20.5±0.2° 2θ, 22.1±0.2° 2θ, 23.4±0.2° 2θ and 25.9±0.2° 2θ measured at a temperature of about 23° C.

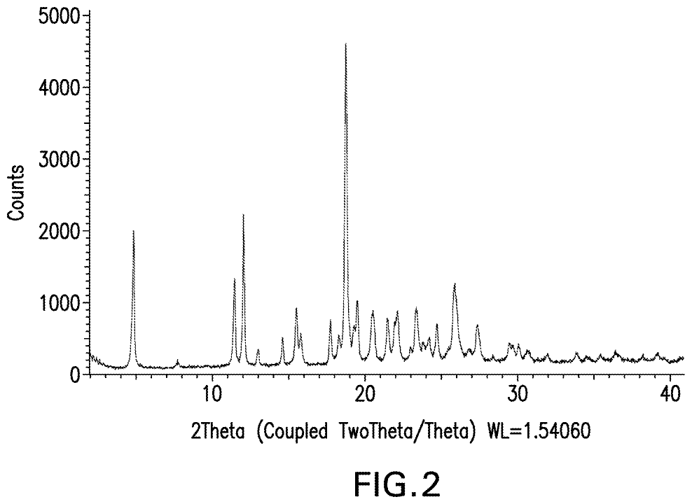

In yet another aspect of the above embodiment, the crystalline Form A of the compound of Formula (I) has an XRPD pattern substantially as shown in FIG. 2.

The crystalline Form A of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide may be characterized thermally. In one embodiment, crystalline Form A of the compound of Formula (I) has a thermal profile measured by Differential Scanning calorimetry (DSC) with a heating rate of 10° C./min comprising an endothermic peak starting at about 186° C. (corresponding to melting). In one embodiment, crystalline Form A of the compound of Formula (I) has a thermal profile measured by Differential Scanning calorimetry (DSC) with a heating rate of 10° C./min comprising an endothermic peak starting at about 3° C. (attributable to loss of residual water) and an endothermic peak starting at about 186°° C. (corresponding to melting).

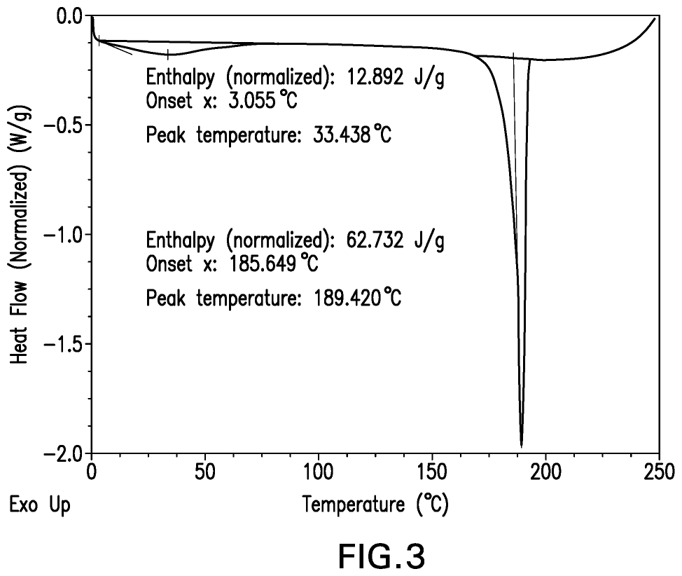

In another embodiment, the crystalline Form A of the compound of Formula (I) has a DSC thermogram that is substantially as shown in FIG. 3. It should be understood that hydrated forms may yield different thermograms (in terms of peak shape and profile) depending on instrument parameters, thus the same material may have thermograms that look substantially different from each other when the data is generated on two different instruments.

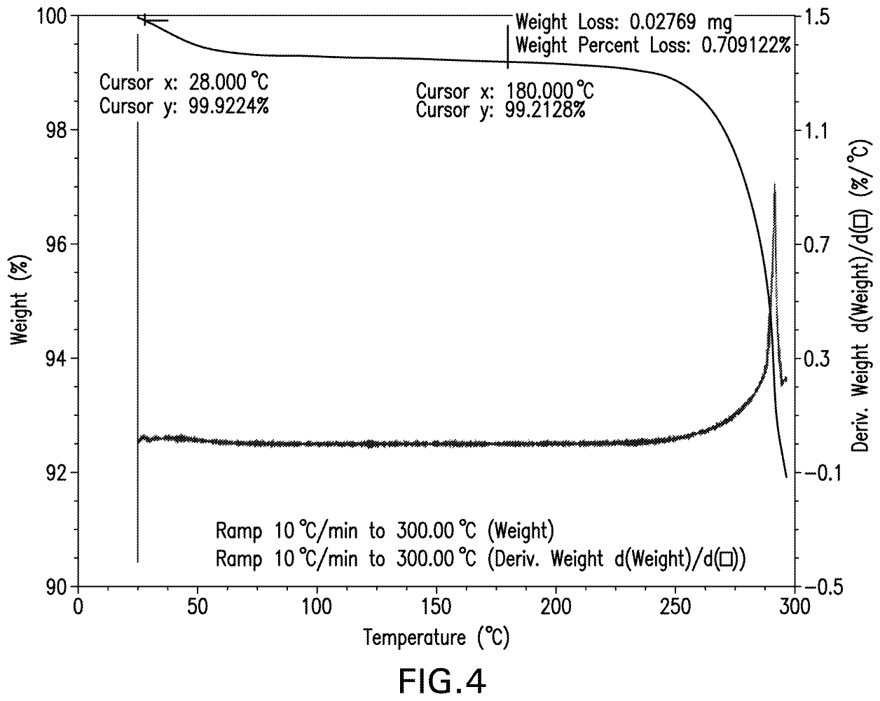

In another embodiment, the crystalline Form A of the compound of Formula (I) has a thermogravimetric analysis (TGA) diagram substantially the same as that shown in shown in FIG. 4. The weight loss by TGA is about 0.7% in the range of about 30° C. to about 180° C.

In yet another embodiment, the crystalline Form A is substantially pure.

In yet another embodiment, the crystalline Form A is substantially chemically pure.

In yet another embodiment, the crystalline Form A is substantially phase pure.

The present invention further provides a crystalline hydrate form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide (Form H_{A}) having an X-ray powder diffraction (XRPD) pattern comprising a representative peak, in terms of 2θ, at 18.8±0.2° 2θ, measured at a temperature of about 23° C. In another embodiment, the XRPD pattern further comprises the additional representative peak 18.6±0.2° 2θ. In one aspect of the previous embodiment, the XRPD pattern for the crystalline Form B may further comprise one, two, three, or four representative peaks chosen from 12.6±0.2° 2θ, 19.3±0.2° 2θ, 22.0±0.2° 2θ and 25.9±0.2° 2θ. In one aspect of the previous embodiment, the XRPD pattern for the crystalline Form B may further comprise the additional representative peak 10.3±0.2° 2θ.

The XRPD pattern for the crystalline hydrate Form H_{A} may comprise one or more (e.g. two, three, four, five or six) representative peaks chosen from 18.8±0.2° 2θ, 18.6±0.2° 2θ, 12.6±0.2° 2θ, 19.3±0.2° 2θ, 22.0±0.2° 2θ, 25.9±0.2° 2θ and 10.3±0.2° 2θ. The XRPD pattern for the crystalline hydrate Form H_{A} may comprise one or more (e.g. two, three, four, five or six) representative peaks selected from the peaks disclosed in table 2 and measured at a temperature of about 23° C.

In another embodiment, said hydrate Form H_{A} is characterized by a x-ray powder diffraction pattern comprising four or more 2θ values selected from the group consisting of 18.8±0.2° 2θ, 18.6±0.2° 2θ, 12.6±0.2° 2θ, 19.3±0.2° 2θ, 22.0±0.2° 2θ, 25.9±0.2° 2θ and 10.3±0.2° 2θ, measured at a temperature of about 23° C.

In another embodiment, said hydrate Form H_{A} is characterized by a x-ray powder diffraction pattern comprising five or more 2θ values selected from the group consisting of 18.8±0.2° 2θ, 18.6±0.2° 2θ, 12.6±0.2° 2θ, 19.3±0.2° 2θ, 22.0±0.2° 2θ, 25.9±0.2° 2θ and 10.3±0.2° 2θ, measured at a temperature of about 23° C.

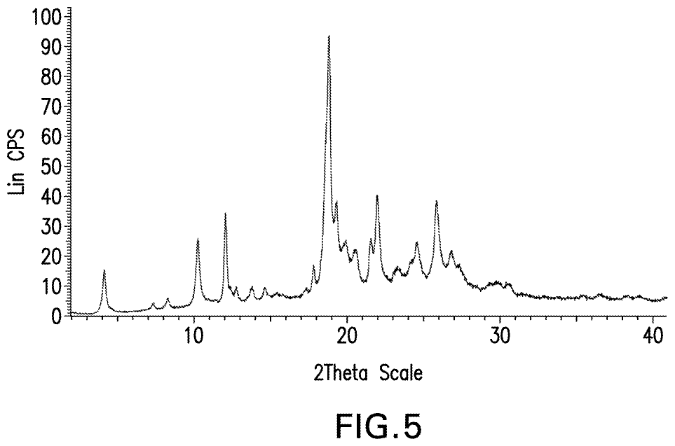

In yet another embodiment, crystalline hydrate Form H_{A} of the compound of Formula (I) has a XRPD pattern substantially as shown in FIG. 5.

The crystalline hydrate Form H_{A} of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide may be characterized thermally.

In one embodiment, crystalline hydrate Form H_{A} of the compound of Formula (I) has a thermal profile measured by Differential Scanning calorimetry (DSC) with a heating rate of 10° C./min, comprising an endothermic peak starting at about 21° C. (attributable to dehydration), an endothermic peak starting at about 142° C. (attributable to melting) and an endothermic peak starting at about 184° C. (corresponding to the melting of hydrate Form H_{A}).

In another embodiment, crystalline hydrate Form H_{A} of the compound of Formula (I) has a thermal profile measured by Differential Scanning calorimetry (DSC) with a heating rate of 10° C./min, comprising an endothermic peak starting at about 17° C. (attributable to dehydration), and an endothermic peak starting at about 181° C. (attributable to melting).

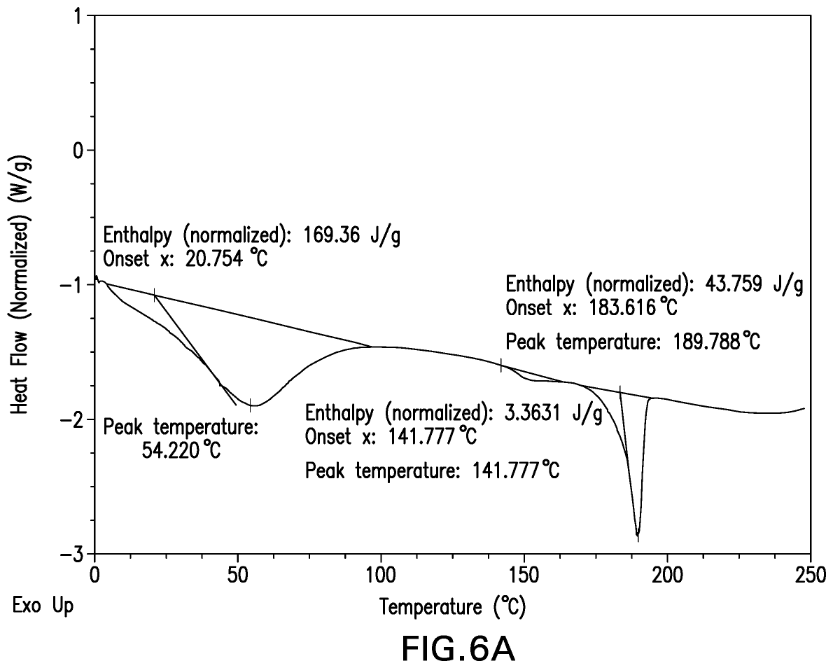

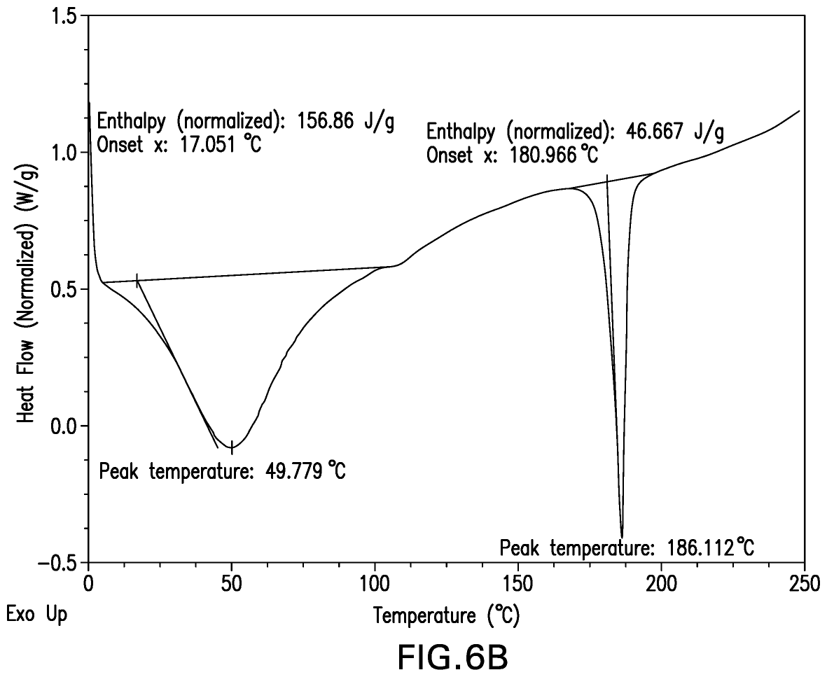

In another embodiment, crystalline hydrate Form H_{A} of the compound of Formula (I) has a DSC thermogram that is substantially as shown in FIG. 6A or FIG. 6B.

It should be understood that hydrated forms may yield different thermograms (in terms of peak shape and profile) depending on instrument parameters, thus the same material may have thermograms that look substantially different from each other when the data is generated on two different instruments.

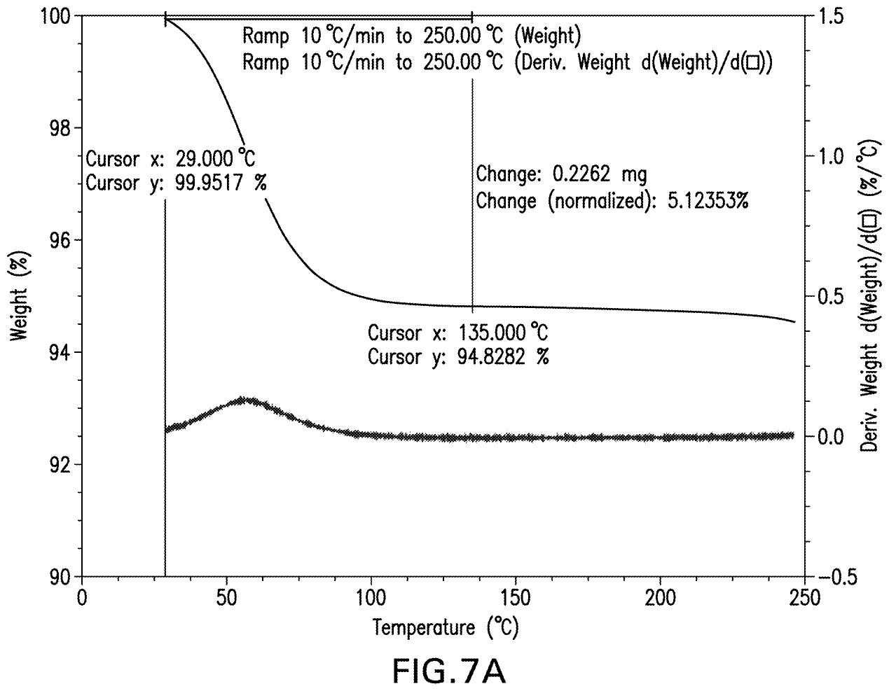

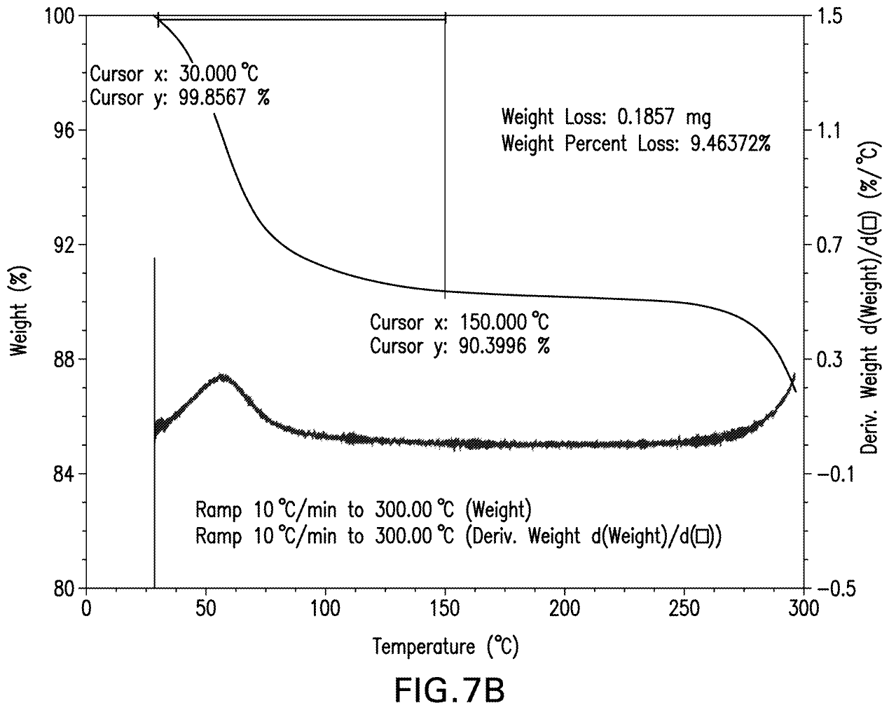

In another embodiment, crystalline hydrate Form H_{A} of the compound of Formula (I) has a thermogravimetric analysis (TGA) diagram substantially the same as that shown in shown in FIG. 7A or in FIG. 7B. The weight loss by TGA is about 5% in the range of 30° C. and 135° C. or 9.5% in the range of 30° C. and 150° C.

In yet another embodiment, the crystalline hydrate Form H_{A} is substantially pure.

In yet another embodiment, the crystalline hydrate Form H_{A} is substantially chemically pure.

In yet another embodiment, the crystalline hydrate Form H_{A} is substantially phase pure.

The present invention further provides a crystalline hydrate form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide (Form H_{B}) having an X-ray powder diffraction (XRPD) pattern comprising a representative peak, in terms of 2θ, at 9.9±0.2° 2θ, measured at a temperature of about 23° C. In another embodiment, the XRPD pattern further comprises the additional representative peak 13.0±0.2° 2θ. In another embodiment, the XRPD pattern further comprises one or more additional representative peaks chosen from 6.5±0.2° 2θ, 11.2±0.2° 2θ, and 12.1±0.2° 2θ. In another embodiment, the XRPD pattern further comprises one or more additional representative peaks chosen from 2.8±0.2° 2θ, and 16.6±0.2° 2θ. In another embodiment, the XRPD pattern further comprises one or more additional representative peaks chosen from 10.6±0.2° 2θ, 16.1±0.2° 2θ and 18.3±0.2° 2θ.

In one aspect of the previous embodiment, the XRPD pattern for the crystalline hydrate Form H_{B} may further comprise one, two, three, or four representative peaks chosen from 2.8±0.2° 2θ, 6.5±0.2° 2θ, 9.9±0.2° 2θ, 10.6±0.2° 2θ, 11.2±0.2° 2θ, 12.1±0.2° 2θ, 13.0±0.2° 2θ, 16.1±0.2° 2θ, 16.6±0.2° 2θ, and 18.3±0.2° 2θ.

The XRPD pattern for the crystalline hydrate Form H_{B} may comprise one or more (e.g. two, three, four, five or six) representative peaks chosen from 2.8±0.2° 2θ, 6.5±0.2° 2θ, 9.9±0.2° 2θ, 10.6±0.2° 2θ, 11.2±0.2° 2θ, 12.1±0.2° 2θ, 13.0±0.2° 2θ, 16.1±0.2° 2θ, 16.6±0.2° 2θ, and 18.3±0.2° 2θ.

The XRPD pattern for the crystalline hydrate Form H_{B} may comprise one or more (e.g. two, three, four, five or six) representative peaks selected from the peaks disclosed in Table 3 and measured at a temperature of about 23° C.

In another embodiment, said hydrate Form H_{B} is characterized by a x-ray powder diffraction pattern comprising four or more 2θ values selected from the group consisting of 2.8±0.2° 2θ, 6.5±0.2° 2θ, 9.9±0.2° 2θ, 10.6±0.2° 2θ, 11.2±0.2° 2θ, 12.1±0.2° 2θ, 13.0±0.2° 2θ, 16.1±0.2° 2θ, 16.6±0.2° 2θ, and 18.3±0.2° 2θ, measured at a temperature of about 23° C.

In another embodiment, said hydrate Form H_{B} is characterized by a x-ray powder diffraction pattern comprising five or more 2θ values selected from the group consisting of 2.8±0.2° 2θ, 6.5±0.2° 2θ, 9.9±0.2° 2θ, 10.6±0.2° 2θ, 11.2±0.2° 2θ, 12.1±0.2° 2θ, 13.0±0.2° 2θ, 16.1±0.2° 2θ, 16.6±0.2° 2θ, and 18.3±0.2° 2θ, measured at a temperature of about 23° C.

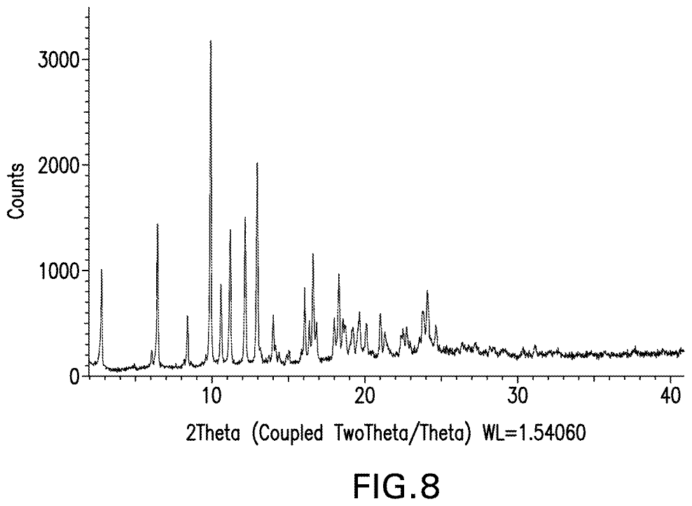

In yet another embodiment, crystalline hydrate Form H_{B} of the compound of Formula (I) has an XRPD pattern substantially as shown in FIG. 8.

The crystalline hydrate Form H_{B} of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide may be characterized thermally. In one embodiment, crystalline hydrate Form H_{B} of the compound of Formula (I) has a differential thermogravimetric profile measured by DSC with a heating rate of 10° C./min, comprising an endothermic peak starting at about 19° C. (attributable to dehydration) and an endothermic peak starting at about 143° C. (attributable to melting).

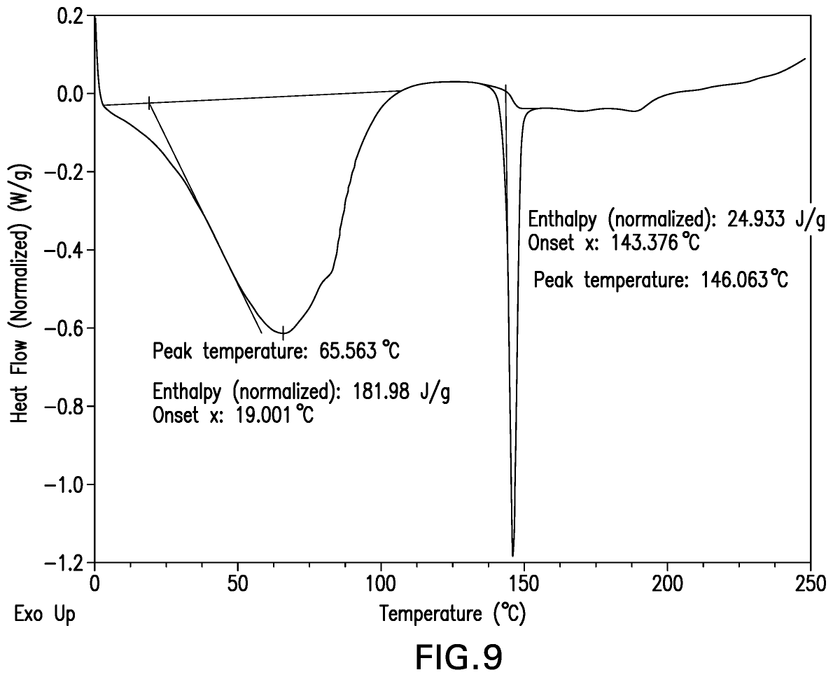

In another embodiment, crystalline hydrate Form H_{B} of the compound of Formula (I) has a DSC thermogram that is substantially as shown in FIG. 9.

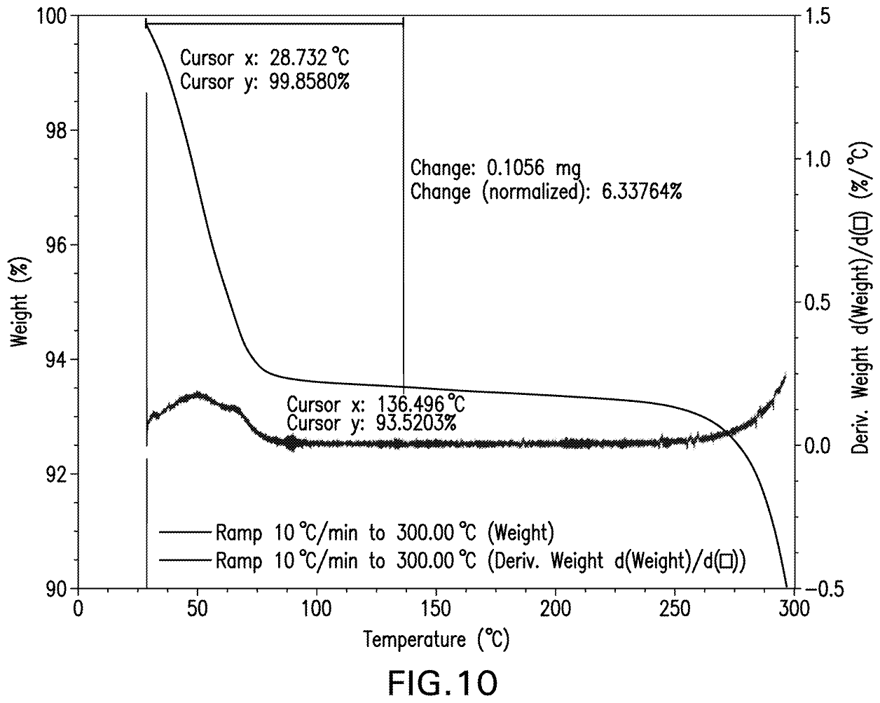

In another embodiment, a crystalline hydrate Form H_{B} has a thermogravimetric analysis (TGA) diagram substantially the same as that shown in shown in FIG. 10. The weight loss by TGA is about 6.3% in the range of about 30° C. to about 136° C.

In yet another embodiment, the crystalline hydrate Form H_{B} is substantially pure.

In yet another embodiment, the crystalline hydrate Form H_{B} is chemically phase pure.

In yet another embodiment, the crystalline hydrate Form H_{B} is substantially phase pure.

### Pharmaceutical Composition, Dosage and Administration

In one embodiment the crystalline forms of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide described herein can be used alone or they can be formulated into a pharmaceutical composition that also contains at least one pharmaceutically acceptable excipient, and often contains at least two or more pharmaceutically acceptable excipients. Excipients may be used that are known in the art without departing from the intent and scope of the present application.

As used herein, the term “pharmaceutically acceptable excipients” includes any and all solvents, carriers, diluents, dispersion media, coatings, surfactants, antioxidants, preservatives (e.g., antibacterial agents, antifungal agents, antioxidants), isotonic agents, absorption delaying agents, salts, drug stabilizers, binders, additives, bulking agents, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). It should be understood that unless a conventional excipient is incompatible with the active ingredient, the use of any conventional excipient in any therapeutic or pharmaceutical compositions is contemplated by the present application.

The pharmaceutical composition can be formulated for particular routes of administration such as topical administration, oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, carriers or buffering agents, as well as adjuvants, such as solvents, preservatives, stabilizers, wetting agents, emulsifiers and bulking agents, etc.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 10-50 mg of active ingredients. Preferably, the pharmaceutical composition or combination of the present invention can be in unit dosage of about 10 mg, about 25 mg or about 50 mg. The therapeutically effective dosage or amount of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys, pigs or isolated tissues and preparations thereof.

In an embodiment of the disclosure, a crystalline form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide (particularly Form A) may be used to prepare pharmaceutical compositions (in particular topical compositions, such as in the form of a cream) which comprise one or more pharmaceutically acceptable excipients.

### Combination

The crystalline form of compound of Formula (I) of the invention (in particular Form A) may be used for the preparation of a medicament which may be administered either simultaneously with, or before or after, one or more other therapeutic agents. The medicament may be administered separately, by the same or different route of administration, or the other agents may be prepared together in the same pharmaceutical composition as that prepared with a crystalline form of compound of Formula (I) of the invention.

The other thereapeutic agent may be selected from a disease-modifying antirheumatic drug (DMARD); vitamin D derivatives; steroids; retinoids; JAK/TYK inhibitors; salicyclic acid; coal tar; anthralin; a calcineurin inhibitor; a modulator of lymphocyte recirculation; a mTOR inhibitor; an ascomycin having immuno-suppressive properties; immunosuppressive compounds; adhesion molecule inhibitors; a chemotherapeutic agent; UV therapy agent; an anti tumor necrosis factor (TNF) agent; T-cell signal inhibitors; blockers of pro-inflammatory cytokines; chemokines blokers; pyrimidine synthesis inhibitors; antimalarials; lymphocytes interacting compounds; sphingosine-1-phosphate (S1P) inhibitors; nonsteroidal anti-inflammatory drugs (“NSAIDs”); an anti-infectious agent; or a acetylsalicylic acid drugs (ASA) including aspirin.

Suitable disease-modifying antirheumatic drugs (DMARDs) for use in the combination include, but are not limited to, gold salts, sulphasalazine, antimalarias, methotrexate, D-penicillamine, azathioprine, mycophenolic acid, cyclosporine A, tacrolimus, sirolimus, minocycline, leflunomide, glococorticoids.

Suitable JAK/TYK inhibitors for use in the combination include, but are not limited to, pan JAK inhibitors or more specific JAK/TYK2 inhibitors such as tofacitinib, baricitinib; JAK inhibitors such as Ruxolitinib.

Suitable calcineurin inhibitors for use in the combination include, but are not limited to, cyclosporin A, FK 506, or FKBP12.

Suitable modulator of lymphocyte recirculation for use in the combination include, but are not limited to, FTY720, or FTY720 analogs.

Suitable mTOR inhibitor for use in the combination include, but are not limited to, rapamycin, 40-O-(2-hydroxyethyl)-rapamycin (everolimus, WO2005/052187), sirolimus, temsirolimus (CCI779), ABT578, AP23573, or TAFA-93.

Suitable ascomycin having immuno-suppressive properties for use in the combination include, but are not limited to ABT-281, ASM981, corticosteroids, cyclophosphamide, azathioprene, methotrexate, leflunomide, mizoribine, mycophenolic acid, mycophenolate mofetil, or 15-deoxyspergualine.

Suitable immunosuppressive compounds for use in the combination include, but are not limited to, a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y; corticosteroids; azathioprin; methotrexate; cyclosporine; retinoids; phosphodiesterase type 4 (PDE4) inhibitors such as apremilast.

Suitable adhesion molecule inhibitors for use in the combination include, but are not limited to, LFA-1 antagonists, ICAM-1 or -3 antagonists, VCAM-4 antagonists, or VLA-4 antagonists.

Suitable chemotherapeutic agent for use in the combination include, but are not limited to, paclitaxel, gemcitabine, cisplatinum, doxorubicin, or 5-fluorouracil.

Suitable UV therapy agent for use in the combination include, but are not limited to, sunlight; or Laser.

Suitable anti-TNF agents for use in the combination include, but are not limited to, monoclonal antibodies to TNF, e.g. infliximab, adalimumab, CDP870; or receptor constructs to TNF-RI or TNF-RII, e.g. Etanercept, PEG-TNF-RI; or TNF-alpha blockers such as adalimumab, infliximab, golimumab.

Suitable T-cell signal inhibitors for use in the combination include, but are not limited to, abatacept, intergrin blockers such as vedolizumab.

Suitable blockers of proinflammatory cytokines for use in the combination include, but are not limited to, IL-1 blockers such as canckinumab; IL-1 trap such as AAL160, ACZ 885; IL-6 blockers; IL-1 receptor blockers such as anakinra; IL-12 inhibitors such as ustekinumab; IL-23 inhibitors such as risankizumab; IL-17 inhibitors such as secukinumab or ixekizumab; or IL17R blockers, such as brodalumab.

Suitable chemokines blockers for use in the combination include, but are not limited to, e.g inhibitors or activators of proteases, e.g. metalloproteases; anti-IL-15 antibodies; anti-IL-6 antibodies such as tocilizumab; or anti-CD20 or anti-CD40 antibodies such as rituximab, iscalimab.

Suitable pyrimidine synthesis inhibitors for use in the combination include, but are not limietd to, leflunomide.

Suitable antimalarials for use in the combination include, but are not limietd to, chloroquin; or hydroxachloroquine

Suitable lymphocytes interacting compounds for use in the combination include, but are not limited to, D-penicillamine.

Suitable sphingosine-1-phosphate (S1P) inhibitors for use in the combination include, but are not limited to, minocycline; or tretracycline.

Suitable NSAIDs for use in the combination include, but are not limited to, Aceclofenac, acemetacin, acetylsalicylic acid, alclofenac, alminoprofen, amfenac, Ampiroxicam, Antolmetinguacil, Anirolac, antrafenine, azapropazone, benorylate, Bermoprofen, bindarit, bromfenac, bucloxic acid, Bucolom, Bufexamac, Bumadizon, butibufen, Butixirat, Carbasalatcalcium, carprofen, choline magnesium trisalicylate, celecoxib, Cinmetacin, Cinnoxicam, clidanac Clobuzarit Deboxamet, dexibuprofen, Dexketoprofen, diclofenac, diflunisal, droxicam, Eltenac, Enfenaminsaure, Etersalat, etodolac, etofenamate, etoricoxib, Feclobuzon, felbinac, fenbufen, fenclofenac, fenoprofen, fentiazac, Fepradinol, Feprazon, Flobufen, floctafenine, flufenamic acid, flufenisal, Flunoxaprofen, flurbiprofen, Flurbiprofenaxetil, Furofenac, Furprofen, Glucametacin, ibufenac, ibuprofen, Indobufen, indomethacin, Indometacinfarnesil, indoprofen, Isoxepac, Isoxicam, ketoprofen, ketorolac, lobenzarit, Lonazolac, lornoxicam, Loxoprofen, lumiracoxib, meclofenamic, Meclofen, mefenamic acid, meloxicam, mesalazine, Miro Profen, Mofezolac, nabumetone, naproxen, niflumic acid, olsalazine, oxaprozin, Oxipinac, oxyphenbutazone, parecoxib, phenylbutazone, Pelubiprofen, Pimeprofen, Pirazolac, Priroxicam, pirprofen, Pranoprofen, Prifelon, Prinomod, Proglumetacin, Proquazon, Protizininsaure, rofecoxib, Romazarit, salicylamide, salicylic acid, Salmi Stein, Salnacedin, salsalate, sulindac, sudoxicam, suprofen, Talniflumate, tenidap, Tenosal, tenoxicam, tepoxalin, tiaprofenic acid, Taramid, Tilnoprofenarbamel, timegadine, Tinoridin, Tiopinac, tolfenamic acid, tolmetin, Ufenamat, valdecoxib, Ximoprofen, zaltoprofen, Zoliprofen and combinations thereof.

Suitable acetylsalicylic acid drugs (ASA) for use in the combination include, but are not limited to, 5-ASA derivatives, such as sulfasalazine.

### Therapeutic kits

In one embodiment, the invention provides a kit comprising two or more separate pharmaceutical compositions, at least one of which contains a medicament prepared using a crystalline form of the compound of Formula (I) (in particular Form A). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

### ABBREVIATIONS

Abbreviations used in the following examples and elsewhere herein are:
AC Active control
aq. Aqueous
AcOH Acetic acid
BINAP racemic 2,2′-bis(diphenylphosphino)-1, 1′-binaphthyl
BOC tertiary butyloxycarbonyl
BP reaction Gene integration reaction between the attB and the attP sites of a Gateway® Cloning gene vector
br. Broad
d doublet
dd doublet of doublets
DCM dichloromethane
DMF N,N-dimethylformamide
DMSO dimethylsulfoxide
EtOAc ethyl acetate
eq Equivalent(s)
GC-MS Gas chromatography and mass spectrometry
h hour(s)
HATU 2-(3H-[1,2,3]triazolo[4,5-b]pyridin-3-yl)-1,1,3,3-tetramethyluronium hexafluorophosphate
HPLC high pressure liquid chromatography
Hz Hertz
IC50 Half maximal inhibitory concentration
J Coupling constant
Liter/milliliter/microliter L/mL/μL
LC-MS liquid chromatography and mass spectrometry
M/mM/μM/nM/pM Molar/millimolar/micromolar/nanomolar/picomolar
MeOH methanol
mCPBA meta-chloroperoxybenzoic acid
MS mass spectrometry
m multiplet
mg/μg milligram/microgram
min minutes
mL milliliter
mmol millimol
m/z mass to charge ratio
NMR nuclear magnetic resonance
ppm parts per million
Prep HPLC Preparatory HPLC
Rt retention time
RT room temperature
S singlet
sat. saturated
SFC Supercritical fluid chromatography
TBME, MTBE methyl tert-butyl ether

NMR measurements were performed on a Bruker 600 MHz or a Bruker 400 MHZ NMR spectrometerusing or not tetramethylsilane (TMS) as an internal standard. Chemical shifts (δ-values) are reported in ppm downfield from tetramethylsilane (TMS), spectra splitting 5 patterns are designated as singlet(s), doublet (d), doublet of doublets (dd), doublet of triplets (dt), doublet of quartets (dq), triplet (t), triplet of doublets (td), quartet (q), quartet of doublets (qd), quintet (quint), septet (sep), multiplet, unresolved or overlapping signals (m), broad signal (br). Deuterated solvents are given in parentheses and have a chemical shifts of dimethyl sulfoxide (δ 2.50 ppm), methanol (δ 3.31 ppm), chloroform (δ 7.26 ppm), or other solvent as indicated in NMR spectral data.

Mass Spectrometry was performed with LC-MS, SFC-MS, or GC-MS systems using electrospray, chemical and electron impact ionization methods from a range of instruments of the following configurations: Agilent 1100 HPLC systems with an Agilent 6110 Mass Spectrometer. [MH]+ refers to protonated molecular ion of the chemical species.

### MS Methods

### Method 1

Column: Ascentis® Express C18 2.7 μm; 2.1×50 mm
Column Temperature: 80° C.
Gradient: from 1 to 50% B in 1.4 min; 50-98% B in 0.30 min Flow: 1.0 mL/min
Eluent A: water+4.76% isopropanol+0.05% FA+3.75 mM AA
Eluent B: isopropanol+0.05% FA

### Method 2

Column: Acquity UPLC BEH C18, 1.7 μm, 2.1×50 mm
Column Temperature: 80° C.
Eluents A: water+0.05% FA+3.75 mM AA, B: isopropanol+0.05% FA
Flow Rate 0.6 mL/min
Gradient from 5 to 98% B in 1.7 min

### Method 3

Column: Acquity UPLC BEH C18, 1.7 μm, 2.1×50 mm
Column Temperature: 80° C.
Eluents A: water+0.05% FA+3.75 mM AA, B: isopropanol+0.05% FA
Flow Rate 0.4 mL/min
Gradient from 5 to 60% B in 8.4 min, then from 60 to 98% B in 1 min

### Preparation of Amorphous Form of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide

To a solution of 3-(4-((S)-2-amino-2-((S)-1,2,3,4-tetrahydronaphthalen-1-yl)acetamido)phenyl)-2,4-dimethylpyridine 1-oxide (Intermediate 3-1; 1.10 g, 2.46 mmol) in DMF (15 mL) were added 1-methyl-1H-pyrazole-5-carboxylic acid (530 mg, 4.2 mmol) followed by HATU (1.9 g, 4.93 mmol), and finally triethylamine (1.7 mL, 12.3 mmol). After stirring at RT overnight, the reaction mixture was diluted with EtOAc (200 mL) and extracted with sat. aq. NaHCO₃, water (twice) and with brine. The organic phase was dried (Na₂SO₄) and evaporated. The residue was purified by column chromatography on silica (column: RediSep 80 g; eluent: TBME/MeOH 100:0 to 0:100) to yield a pale yellow solid which was triturated with DCM/MeOH 4:1 at 0° C., followed by filtering and washing the solid with cold isopentane to yield the title compound (771 mg, 62%) as a colorless solid.

LC-MS: Rt=4.48 min; MS: m/z=510 [MH]+ (Method 3).

1H NMR (400 MHZ, DMSO-d6) δ 10.57 (s, 1H), 8.95 (d, 1H), 8.20 (d, 1H), 7.83 (d, 2H), 7.46 (d, 1H), 7.40 (d, 1H), 7.28-7.16 (m, 3H), 7.11-7.05 (m, 3H), 7.05-6.94 (m, 1H), 4.82 (dd, 1H), 3.90 (s, 3H), 3.38-3.34 (m, 1H), 2.93-2.71 (m, 2H), 2.23-2-12 (br. m, 1H), 2.09 (s, 3H), 1.97 (s, 3H), 1.85-1.65 (m, 3H) ppm.

The XRPD spectrum was measured at room temperature (about 296 K) on a Bruker D8 Discover new instrument using a VANTEC-500 detector. The radiation used for collecting the data was Cu (λ=1.5418 Å). The X-ray generator power was 40 kV, 40 mA, 1600. The Step size, resolution was time/step 60 s, steps 2. Diffraction data were collected in the range 9 to 36° (2 Theta value). The scan time was 120 s.

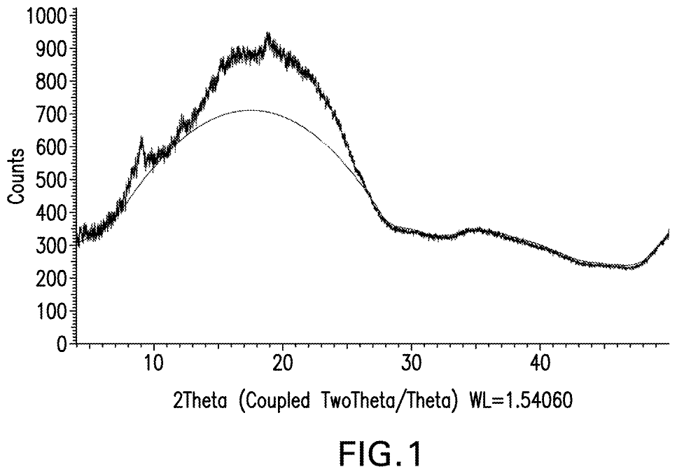

The obtained XRPD spectrum is shown in FIG. 1 which shows that the obtained compound, 4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide, was amorphous.

### Intermediate 3-1: 3-(4-((S)-2-amino-2-((S)-1,2,3,4-tetrahydronaphthalen-1-yl)acetamido)phenyl)-2,4-dimethylpyridine 1-oxide

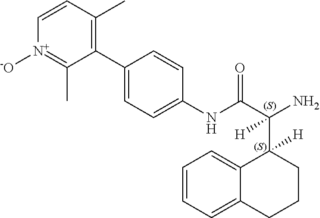

Step 1: 3-(4-((S)-2-((tert-butoxycarbonyl)amino)-2-((S)-1,2,3,4-tetrahydronaphthalen-1-yl)acetamido)phenyl)-2,4-dimethylpyridine 1-oxide

To a solution of 3-(4-aminophenyl)-2,4-dimethylpyridine 1-oxide (Intermediate 1-1; 1.26 g, 5.89 mmol) in DMF (30 mL) were sequentially added(S)-2-((tert-butoxycarbonyl)amino)-2-((S)-1,2,3,4-tetrahydronaphthalen-1-yl)acetic acid (Intermediate 2-1, compound of peak 3; 1.50 g, 4.91 mmol), HATU (2.24 g, 5.89 mmol) and Et₃N (2.04 mL, 14.74 mmol). The mixture was stirred at RT for 18h, then diluted with EtOAc and washed with sat. aq. NaHCO₃, water, and brine. The organic phase was dried (Na₂SO₄) and all volatiles were evaporated under reduced pressure. The residue was purified by column chromatography on silica (eluent: 0% to 25% TBME in MeOH) to yield the title compound (1.97 g, 80%) as a slightly yellow solid.

LC-MS: Rt=1.08 min; MS m/z=502.3 [MH]+ (Method 1).

Step 2: 3-(4-((S)-2-amino-2-((S)-1,2,3,4-tetrahydronaphthalen-1-yl)acetamido)phenyl)-2,4-dimethylpyridine 1-oxide

To a solution of 3-(4-((S)-2-((tert-butoxycarbonyl)amino)-2-((S)-1,2,3,4-tetrahydronaphthalen-1-yl)acetamido)phenyl)-2,4-dimethylpyridine 1-oxide (1.97 g, 3.93 mmol) in dioxane (40 mL), was added dropwise 4M HCl in dioxane (40 mL) and the solution stirred for 18 h at RT. All volatiles were removed under reduced pressure. The residue was diluted with 1M aq. Na₂CO₃ and extracted three times with DCM/isopropanol 4:1. The combined organic phases were dried (Na₂SO₄) and concentrated. The residue was purified by column chromatography on silica (0% to 10% TBME in MeOH) to yield the title compound (1.47 g, 91%) as a white foam. LC-MS: Rt=0.62 min; MS m/z=402.2 [MH]+ (Method 1).

### Intermediate 1-1:3-(4-aminophenyl)-2,4-dimethylpyridine 1-oxide

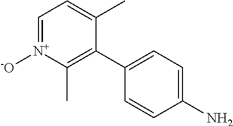

Step 1: 3-bromo-2,4-dimethylpyridine 1-oxide

To a solution of 3-bromo-2,4-dimethylpyridine (80 g, 0.430 mol) in DCM (800 mL) was added m-CPBA (90 g, 0.516 mol, 1.2 eq) at 10˜15° C. After stirring for 3 hours at the same temperature HPLC analysis indicated complete conversion of the starting material. The mixture was poured into 10% Na₂SO₃ aqueous solution, stirred for 5 minutes and the layers were separated. The aqueous phase was extracted with DCM (400 mL). The organic phases were combined and washed with 10% aqueous Na₂CO₃. The aqueous phase was re-extracted with DCM (400 mL). The organic phases were combined, washed twice with brine (400 mL), dried (Na₂SO₄) and concentrated to yield the title compound (68 g, 78% yield) as an off-white solid.

1H NMR (400 MHZ, DMSO) δ 8.22 (d, 1H), 7.30 (d, 1H), 2.57 (s, 3H), 2.34 (s, 3H) ppm.

Step 2: 3-(4-aminophenyl)-2,4-dimethylpyridine 1-oxide

To a solution of 3-bromo-2,4-dimethylpyridine 1-oxide (70 g, 0.346 mol) in dioxane (700 mL) were added 4-(4,4,5,5-tetramethyl-1,3,2-dioxaborolan-2-yl) aniline (91 g, 0.416 mol), Na₂CO₃ (110 g, 1.038 mol), water (140 mL) and Pd(dppf)Cl2·DCM complex (12.7 g, 0.017 mol). The reaction mixture was purged with nitrogen, and stirred at 100° C. for 16 hours. HPLC analysis indicated complete conversion of the starting material. The reaction mixture was filtered, the filtrate was concentrated, taken-up with DCM (700 mL) and washed with brine (350 mL). The aqueous phase was extracted with DCM (350 mL×5). The organic phases were combined, dried (Na₂SO₄) and concentrated. The crude product was purified by column chromatography on silica (DCM:MeOH 15:1) to yield the title compound (53.4 g, 72% yield) as a light brown solid.

1H NMR (400 MHZ, DMSO-d6) δ 8.14 (d, J=6.6 Hz, 1H), 7.17 (d, J=6.6 Hz, 1H), 6.84 (d, J=8.3 Hz, 2H), 6.66 (d, J=8.3 Hz, 2H), 5.26 (s, 2H), 2.11 (s, 3H), 1.99 (s, 3H) ppm.

### Intermediate 2-1: (S)-2-((tert-butoxycarbonyl)amino)-2-((S)-1,2,3,4-tetrahydronaphthalen-1-yl)acetic acid

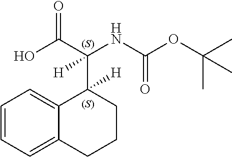

To a cooled (0° C.) mixture of 2-(1,2,3,4-tetrahydronaphthalen-1-yl)glycine (2 g, 9.74 mmol) in a mixture of dioxane (30 mL) and 1M aq. NaOH (15 mL) was added di-tert.-butyl-dicarbonate (3.4 g, 14.6 mmol). After stirring for 5 h at RT, the mixture was partitioned between EtOAc and aq. 10% KHSO₄. The organic layer was washed with water and brine, dried (Na₂SO₄) and evaporated. The residue was triturated with DCM/methanol 1:1, the resulting solid was filtered and dried in vacuo. The mother liqour was purified by column chromatography on silica (column: RediSep 40 g; elution with 5 to 70% EtOAc in heptane). Pure fractions were evaporated and combined with the filtered material to yield a stereoisomeric mixture of the title compound (2.37 g, 76%) as a colorless solid.

The separation of the individual stereoisomers was achieved by chiral SFC (column: ChiralPak AY, 300×50 mm I.D., 10 μm; 25% ethanol, flow: 100 mL/min.

Analytical chiral SFC (ChiralPak AY, 150×4.6 mm I.D., 3 μm, 5-40% ethanol+0.05% DEA; flow=2.5 mL/min, T=35° C.):
Peak 1: Rt=2.41 min: 873 mg
Peak 2: Rt=2.99 min: 130 mg
Peak 3: Rt=3.29 min: 1040 mg
Peak 4: Rt=4.71 min: 78 mg

LC-MS: Rt=5.05 min; MS m/z=304.2 [M-H]+ (Method 2).

The stereochemistry of the eluted compound of peak 3 (designated intermediate 2-1) was determined as (S,S) by X-ray crystallography.

### Preparation of crystalline and hydrate forms of 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent or solvent mixture include, for example, evaporation of the solvent, decreasing the temperature of the solvent or solvent mixture, crystal seeding a supersaturated solvent mixture of the molecule and/or salt, freeze drying the solvent mixture, and addition of antisolvents (countersolvents) to the solvent mixture. Exemplary methods of preparing the crystalline forms described herein are set forth in detail below.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Indiana (1999).

For crystallization techniques that employ solvents, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature. This may also be referred to as a suspension.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369-377. In general, seeds of small size are needed to control effectively the growth of crystals in the batch. Seeds of small size may be generated by sieving, milling, or micronizing large crystals, or by micro-crystallization of solutions. Care should be taken that milling or micronizing of crystals does not result in any change in crystallinity form the desired crystal form (i.e., change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as solid state nuclear magnetic resonance, differential scanning calorimetry, x-ray powder diffraction, or the like, to assure formation of the preferred crystalline form of the product. The resulting crystalline form is typically produced in an amount of greater than about 70 weight % isolated yield, preferably greater than 90 weight % isolated yield, based on the weight of the compound originally employed in the crystallization procedure. The product may be co-milled or passed through a mesh screen to delump the product, if necessary.

Alternatively, crystalline forms may be prepared directly from the reaction medium of the final process for preparing 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide. This may be achieved, for example, by employing in the final process step a solvent or a mixture of solvents from which 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide may be crystallized. In addition, crystalline forms may be obtained by distillation or solvent addition techniques.

In addition to the methods discussed briefly below, it should be understood that various analytical methods may be used for the characterization of any of the materials described herein.

The following non-limiting examples are illustrative of the disclosure.

### EXAMPLES

### Example 1: Preparation of the Crystalline Form A

### Step 1

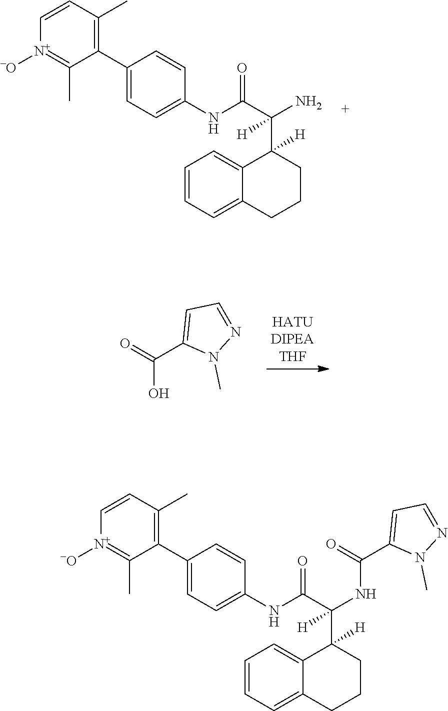

To a solution of 1-methyl-1H-pyrazole-5-carboxylic acid (18.341 g, 145.4 mmol) in THF (330 ml) was added O-(7-azabenzotriazol-1-yl)-N,N,N′,N′-tetramethyluronium Hexafluorophosphate (HATU) (63.197 g, 166.2 mmol) followed by N,N-Diisopropylethylamine (DIPEA) (53.703 g, 415.5 mmol) at 20 to 30°° C. The mixture was stirred at 20 to 30° C. for 0.5 h. After that, a solution of 3-(4-((S)-2-amino-2-((S)-1,2,3,4-tetrahydronaphthalen-1-yl)acetamido)phenyl)-2,4-dimethylpyridine 1-oxide in THF (166 g, 33.5% w/w, 138.5 mmol) was added into the mixture. The reaction mixture was stirred at 20 to 30° C. for 3 h. The reaction was quenched by addition of water (330 ml) at 20 to 30° C. The solution was then extracted with EtOAc (330 ml, 2×). The organic phase was washed with 1N HCl aq. (330 ml, 2×), 10% NaHCO₃ aq. (330 ml, 2×) and water (330 ml), then concentrated under vacuum. The crude residue was charged into MTBE (500 ml) and then stirred for a while. After filtration, the wet cake was dissolved in DCM (500 ml) and MeOH (50 ml). The solution was washed with 10% NH₃·H₂O (500 ml, 3×) and water (500 ml), and then concentrated under vacuum to obtain the crude product. The crude product was purified by slurry in MeOH (150 ml) and MTBE (500 ml) at 20 to 30° C. overnight. The product was further charged into water (500 ml) and then stirred at 65 to 70° C. for 2 h. After filtration and drying, 38.9 g of pale-brown solid was obtained.

### Step 2

A suspension of 1.5 g of the material obtained according to the above procedure was equilibrated in water at 25° C. for one week. Afterwards, the solid was separated by filtration and was dried at 30° C. under vacuum overnight (about 18 hours). The obtained material was characterized by high resolution XRPD, TGA and DSC showing that crystalline Form A was obtained.

### Example 1A: High-Resolution Powder X-Ray Diffraction of Crystalline Form A

The sample material from Example 1, Step 2, was filled into a silicon low-background specimen holder for reflection measurements and the filled holder was placed in the diffractometer. XRPD patterns were collected at room temperature (about 296 K) on a Bruker D8 Advance system equipped with LynxEye solid state detector in reflection mode. The radiation used for collecting the data was CuKα (λ=0.15418 nm). Diffraction data were collected in the range 2-40° 2θ and peaks resulting from CuKα1 radiation (λ=1.5406 Å) were evaluated. The obtained spectrum is shown in FIG. 2 and the peaks are listed in Table 1.

TABLE 1
X-ray powder diffraction data for crystalline Form A

| 2θ [°] | d value [Å] | Intensity [%] |
|---|---|---|
| 4.87 | 18.13 | 43% |
| 11.46 | 7.72 | 27% |
| 12.06 | 7.33 | 47% |
| 13.01 | 6.80 | 5% |
| 14.62 | 6.05 | 9% |
| 15.51 | 5.71 | 17% |
| 15.81 | 5.60 | 10% |
| 17.76 | 4.99 | 14% |
| 18.32 | 4.84 | 8% |
| 18.76 | 4.73 | 100% |
| 19.33 | 4.59 | 10% |
| 19.51 | 4.55 | 18% |
| 20.54 | 4.32 | 16% |
| 21.51 | 4.13 | 12% |
| 22.05 | 4.03 | 12% |
| 22.14 | 4.01 | 15% |
| 23.37 | 3.80 | 16% |
| 24.23 | 3.67 | 7% |
| 24.73 | 3.60 | 11% |
| 25.89 | 3.44 | 22% |
| 27.40 | 3.25 | 11% |
| 29.52 | 3.02 | 5% |
| 30.10 | 2.97 | 5% |

all peaks with relative intensity ≥5%

### Example 1B: Differential Scanning Calorimetry (DSC) of Form A

The DSC thermogram was recorded with a TA Discovery DSC instrument using heat flux compensation (TA Instruments, USA). The TA Discovery instrument was calibrated for temperature and enthalpy according to the manufacturer's instructions using certified reference substances like indium. About 2 mg of sample material was sealed in a standard pin-holed aluminum pan and heated in the DSC from 0° C. to 250° C., at a heating rate of 10° C./min. Dry N2 gas, at a flow rate of 50 ml/min was used to purge the DSC equipment during the measurement. The obtained DSC thermogram is shown in FIG. 3. Two thermal events were observed:
First endotherm (attributable to loss of residual water): T onset=3.1° C., ΔH=13 J/g.
Second endotherm (attributable to melting): T onset=185.6° C., ΔH=63 J/g.

### Example 1C: Thermogravimetric Analysis (TGA) of Form A

Thermogravimetric analysis was performed using a TA Discovery TGA instrument (TA Instruments, USA). About 2-10 mg of sample material was sealed in a standard aluminum pan with pierced lid and heated in the TGA from 30° C. to 300° C., at a heating rate of 10° C./min. Dry N₂ gas, at a flow rate of 20 ml/min was used to purge the TGA equipment during the measurement. The obtained graph is shown in FIG. 4. Loss on drying between about 30° C. and 180° C. was 0.7% m/m.

### Example 2: Preparation of Hydrate Form H_{A}

### Method 1

The material obtained from Example 1, Step 1, was stored at 92% RH at room temperature for three days and was analyzed by XRPD directly afterwards. Hydrate H_{A} was observed as a result of this storage.

### Method 2

In a separate experiment, about 50 mg of the material from Example 1, Step 1, was equilibrated in 0.5 mL of a water/acetonitrile mixture containing 7.1% m/m water at 25° C. for two weeks. Afterwards, the solid phase was separated by filtration and analyzed by XRPD. The XRPD pattern of the wet solid cake showed Hydrate H_{A}.

### Example 2A: High-Resolution Powder X-Ray Diffraction of Hydrate Form H_{A}

The sample material was filled into a silicon low-background specimen holder for reflection measurements and the filled holder was placed in the diffractometer. XRPD patterns were collected at room temperature (about 296 K) on a Bruker D8 Advance system equipped with LynxEye solid state detector in reflection mode. The radiation used for collecting the data was CuKα (λ=0.15418 nm). Diffraction data were collected in the range 2-40° 2θ and peaks resulting from CuKα1 radiation (λ=1.5406 Å) were evaluated.

The obtained spectrum using a sample of material from Method 1 is shown in FIG. 5 and the peaks are listed in Table 2.

TABLE 2
X-ray powder diffraction data for crystalline hydrate Form H_{A}

| 2θ [°] | d value [Å] | Intensity [%] |
|---|---|---|
| 4.16 | 21.21 | 17% |
| 10.25 | 8.62 | 26% |
| 12.06 | 7.33 | 35% |
| 12.40 | 7.13 | 6% |
| 12.76 | 6.93 | 6% |
| 13.82 | 6.40 | 5% |
| 17.83 | 4.97 | 11% |
| 18.60 | 4.77 | 59% |
| 18.83 | 4.71 | 100% |
| 19.29 | 4.60 | 33% |
| 19.92 | 4.45 | 18% |
| 20.53 | 4.32 | 13% |
| 21.56 | 4.12 | 16% |
| 22.00 | 4.04 | 34% |
| 23.32 | 3.81 | 6% |
| 24.23 | 3.67 | 8% |
| 24.59 | 3.62 | 16% |
| 25.88 | 3.44 | 32% |
| 26.83 | 3.32 | 13% |
| 27.29 | 3.27 | 8% |

all peaks with relative intensity ≥5%

### Example 2B: Differential Scanning Calorimetry (DSC) of Hydrate Form H_{A}

The DSC thermogram was recorded with a TA Discovery DSC instrument using heat flux compensation (TA Instruments, USA). The TA Discovery instrument was calibrated for temperature and enthalpy according to the manufacturer's instructions using certified reference substances like indium. About 2 mg of sample material was sealed in a standard pin-holed aluminum pan and heated in the DSC from 0° C. to 250° C., at a heating rate of 10° C./min. Dry N2 gas, at a flow rate of 50 ml/min was used to purge the DSC equipment during the measurement.

The obtained DSC thermogram using material from Method 1 is shown in FIG. 6A.

Three thermal events were observed:
First endotherm (attributable to dehydration): Tonset=20.8° C., ΔH=169 J/g.
Second endotherm (attributable to melting): Tonset=141.8° C., ΔH=3 J/g.
Third endotherm (attributable to melting): Tonset=183.6° C., ΔH=44 J/g.

The obtained DSC thermogram using material from Method 2 is shown in FIG. 6B.

Two thermal events are observed:
First endotherm (attributable to dehydration): Tonset=17.0° C., ΔH=157 J/g.
Second endotherm (attributable to melting): Tonset=181.0° C., ΔH=47 J/g.

### Example 2C: Thermogravimetric Analysis (TGA) of Hydrate Form H_{A}

Thermogravimetric analysis was performed using a TA Discovery TGA instrument (TA Instruments, USA). About 2-10 mg of sample material was sealed in a standard aluminum pan with pierced lid and heated in the TGA from 30° C. to at least 250° C., at a heating rate of 10° C./min. Dry N₂ gas, at a flow rate of 20 ml/min was used to purge the TGA equipment during the measurement.

The obtained graph using material from Method 1 is shown in in FIG. 7A. Loss on drying between about 30° C. and 135° C. was 5.12% m/m

The obtained graph using material from Method 2 is shown in in FIG. 7B. Loss on drying between about 30° C. and 150° C. was 9.46% m/m

Dynamic vapor sorption (DVS) data obtained separately (not shown) suggests an even higher water content for hydrate H_{A} in the range of 16 to 18%. Due to the unstable nature of hydrate H_{A} at ambient temperature and humidity, water loss occurs quickly during handling of the samples and the high water content observed by DVS analysis was not seen in DSC or TGA analysis. This water loss behavior is considered to explain the different DSC and TGA results for the two hydrate H_{A} samples.

### Example 3: Preparation of Hydrate Form H_{B}

About 50 mg of the material obtained from Example 1, Step 1, was equilibrated in 0.5 mL of a water/acetonitrile mixture containing 7.1% m/m water at 50° C. for at least one week. Afterwards, the solid phase was separated by filtration, the solid was dried in air for about 10 minutes and was analyzed by XRPD afterwards. The XRPD pattern of the solid cake showed Hydrate H_{B}.

### Example 3A: High-Resolution Powder X-Ray Diffraction of Hydrate Form H_{B}

The sample material was filled into a silicon low-background specimen holder for reflection measurements and the filled holder was placed in the diffractometer. XRPD patterns were collected at room temperature (about 296 K) on a Bruker D8 Advance system equipped with LynxEye solid state detector in reflection mode. The radiation used for collecting the data was CuKα (λ=0.15418 nm). Diffraction data were collected in the range 2-40° 2θ and peaks resulting from CuKα1 radiation (λ=1.5406 Å) were evaluated.

The obtained spectrum using a sample of material from Example 3 is shown in FIG. 8 and the peaks are listed in Table 3.

TABLE 3
X-ray powder diffraction data for crystalline hydrate Form H_{B}

| | d value | |
|---|---|---|
| 2θ [°] | [Å] | Intensity [%] |
| 2.82 | 31.33 | 30% |
| 6.10 | 14.48 | 5% |
| 6.49 | 13.61 | 45% |
| 8.42 | 10.49 | 16% |
| 9.93 | 8.90 | 100% |
| 10.61 | 8.33 | 24% |
| 11.21 | 7.89 | 42% |
| 12.19 | 7.25 | 45% |
| 12.97 | 6.82 | 62% |
| 14.02 | 6.31 | 15% |
| 16.09 | 5.50 | 22% |
| 16.39 | 5.40 | 11% |
| 16.64 | 5.32 | 33% |
| 16.86 | 5.25 | 12% |
| 18.02 | 4.92 | 12% |
| 18.32 | 4.84 | 26% |
| 18.66 | 4.75 | 8% |
| 19.21 | 4.62 | 9% |
| 19.67 | 4.51 | 13% |
| 20.11 | 4.41 | 10% |
| 21.03 | 4.22 | 13% |
| 21.34 | 4.16 | 7% |
| 22.52 | 3.94 | 8% |
| 22.75 | 3.91 | 8% |
| 23.84 | 3.73 | 12% |
| 24.12 | 3.69 | 18% |
| 24.67 | 3.61 | 8% |

all peaks with relative intensity ≥5%

### Example 3B: Differential Scanning Calorimetry (DSC) of Hydrate Form H_{B}

The DSC thermogram was recorded with a TA Discovery DSC instrument using heat flux compensation (TA Instruments, USA). The TA Discovery instrument was calibrated for temperature and enthalpy according to the manufacturer's instructions using certified reference substances like indium. About 2 mg of sample material was sealed in a standard pin-holed aluminum pan and heated in the DSC from 0° C. to 250° C., at a heating rate of 10° C./min. Dry N2 gas, at a flow rate of 50 ml/min was used to purge the DSC equipment during the measurement.

The obtained DSC thermogram using material from Example 3 is shown in FIG. 9.

Two thermal events were observed:
First endotherm (attributable to dehydration): Tonset=19.0° C., ΔH=182 J/g.
Second endotherm (attributable to melting): Tonset=143.4° C., ΔH=25 J/g.

### Example 3C: Thermogravimetric Analysis (TGA) of Hydrate Form H_{B}

Thermogravimetric analysis was performed using a TA Discovery TGA instrument (TA Instruments, USA). About 2-10 mg of sample material was sealed in a standard aluminum pan with pierced lid and heated in the TGA from 30° C. to 300° C., at a heating rate of 10° C./min. Dry N₂ gas, at a flow rate of 20 ml/min was used to purge the TGA equipment during the measurement.

The obtained graph using material from Example 3 is shown in in FIG. 10.

Loss on drying between about 30° C. and about 136° C. was 6.34% m/m.

1. A crystalline form of the compound 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide.

2. The crystalline form according to claim 1 of the compound 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide, characterized by an x-ray powder diffraction pattern comprising a representative peak, in terms of 2θ, at 18.8±0.2° 2θ when measured at a temperature of about 23° C. and an x-ray wavelength, λ, of 1.5406 Å.

3. The crystalline form according to claim 1 of the compound 2,4-dimethyl-3-(4-((S)-2-(1-methyl-1H-pyrazole-5-carboxamido)-2-((S)-1,2,3,4-tetrahydronaphthalen-1-yl)acetamido)phenyl)pyridine 1-oxide, characterized by an x-ray powder diffraction pattern comprising one or more representative peaks in terms of 2θ selected from the group consisting of 4.9±0.2° 2θ, 11.5±0.2° 2θ, 12.0±0.2° 2θ, 15.5±0.2° 2θ, 18.8±0.2° 2θ, 19.5±0.2° 2θ, 20.5±0.2° 2θ, 22.1±0.2° 2θ, 23.4±0.2° 2θ and 25.9±0.2° 2θ, when measured at a temperature of about 23° C. and an x-ray wavelength, λ, of 1.5406 Å.

4. The crystalline form according to claim 1 having an x-ray diffraction spectrum substantially the same as the x-ray powder diffraction spectrum shown in FIG. 2.

5. The crystalline form of claim 1, characterized by a differential thermogravimetric profile measured by Differential Scanning calorimetry (DSC) with a heating rate of 10° C./min, comprising an endothermic peak starting at about 186° C.

6. The crystalline form according to claim 1 having a differential scanning calorimetry (DSC) thermogram substantially the same as that shown in FIG. 3.

7. The crystalline form of claim 1, having a weight loss on drying of about 0.7% in the range of about 30° C. to about 180° C., as determined by thermogravimetric analysis.

8. The crystalline form according to claim 1 having a thermogravimetric analysis (TGA) diagram substantially the same as that shown in FIG. 4.

9. A hydrate of the crystalline form claimed in claim 1.
